(19) United States
(12) Patent Application Publication
(10) Pub. No.: US 2005/0142537 A1
(43) Pub. Date: Jun. 30, 2005

(54) System and method for rapid detection of metabolic deviations of a biological sample

(76) Inventors: Ronald Rieder, Medford, MA (US); John R. Howatt, Bedford, MA (US)

Correspondence Address: IANDIORIO & TESKA;INTELLECTUAL PROPERTY LAW ATTORNEYS, 260 BEAR HILL ROAD, WALTHAM, MA, 02451-1018, US

(21) Appl. No.: 10/868,399

(22) Filed: Jun. 15, 2004

Related U.S. Application Data
(60) Provisional application No. 60/532,399, filed on Dec. 24, 2003.

(51) Int. Cl. C12Q 1/70; C12Q 1/68; C12M 1/34

(52) U.S. Cl. 435/5; 435/287.2

(57) ABSTRACT

A system for rapid detection of metabolic deviations of a biological sample including at least one control chamber having spaced electrodes for receiving a biological sample, at least one target chamber having spaced electrodes for receiving the biological sample and a biological reagent, and a measuring and detection device responsive to the control chamber and the target chamber for measuring, at a first time, first impedances of the biological sample in the control chamber and the biological reagent and the biological sample in the target chamber and, at a second time, measuring second impedances of the biological sample in the control chamber and the biological sample and the biological reagent in the target chamber and comparing the second impedances to the first impedances to measure the effect of the biological reagent inducing metabolic deviations to the biological sample in the target chamber and determine whether the biological sample is sensitive to the biological reagent.

### RELATED APPLICATIONS

This application claims priority of U.S. Provisional Application No. 60/532,399, filed Dec. 24, 2003, entitled “Rapid and Robust Method for the Detection of Microbial Growth”.

### FIELD OF THE INVENTION

This invention relates to an improved system and method for the rapid detection of metabolic deviations of a biological sample.

### BACKGROUND OF THE INVENTION

Microbial organisms (e.g., the bacteria Escherichia coli and Staphylococcus aureus) cause infection and disease in millions of patients each year. For example, urinary tract infections (UTI) are some of the most common bacterial infections and accounted for over eight million visits to physicians in 1997 alone. Common treatment for patients symptomatic of a microbial infection is to collect a sample, such as blood, urine, sputum, and the like, for testing to determine the presence of a microbial organism in the sample that causes the illness and prescribe antimicrobial drugs (e.g., antibiotics) based on the test results. Conventional techniques for detecting the presence of a microbial organism in a biological sample typically involve sending the sample to a laboratory where the microbial organism is identified, isolated, and cultured. This approach is very time consuming, typically taking 48 to 72 hours or more to obtain results for fast growing organisms.

Often before laboratory results are obtained a physician dispenses an empirically chosen antimicrobial drug, which is often a broad spectrum antibiotic, in order to provide temporary relief to the patient. However, dispensing empirically chosen antimicrobial drugs is often not the most effective treatment because specific antimicrobial agents or drugs which are more effective against the detected microbial organism often exist. However, determining which antimicrobial drug is the most effective for the detected microbial organism requires additional testing, known as susceptibility or sensitivity testing (discussed below). Moreover, dispensing broad spectrum drugs often results in the generation of new microbial organisms that are resistant to the drugs, which requires new drugs to be developed which is expensive and time consuming.

Conventional susceptibility or sensitivity testing systems and methods determine the most effective biological reagent (e.g., antimicrobial agent or drug) for the identified microbial organisms by monitoring the viability of the organism after exposure to antimicrobial agents. Conventional sensitivity testing methods include, inter alia, disk diffusion tests, broth microdilution tests, agar microdilution tests, and agar gradient methods in the biological sample. These complicated tests must be performed by a skilled technician in a laboratory and take an additional 12 to 24 hours or more to complete after the microbial organism has been isolated and cultured. Because these systems and methods are very time consuming, the physician does not always utilize these tests and, as discussed above, often administers an empirically chosen antimicrobial drug. The result is that the most effective antimicrobial drug is often not prescribed which may result in patient relapse, increased costs, and the progression of resistance of microbial organisms to the antimicrobial drugs.

Moreover, conventional susceptibility or sensitivity systems and methods are not traditionally used to determine the sensitivity of the biological sample to viruses.

Other conventional methods, such as impedance based detection, can be used to monitor the viable growth of microbial organisms in a biological sample by measuring the changes in the dielectric properties of a suspension of the biological sample (e.g., conductance, capacitance, and/or the full impedance) which result from the metabolic activity of the microorganisms in the biological sample. Metabolic activities (deviations) of the microorganisms include, inter alia, cellular metabolic end-products, cellular growth, change in cell mass from binary fission, changes in surface morphology, and synthesis of highly charged molecules, such as DNA, proteins, amino acids, and the like. However, conventional impedance based detection devices and methods rely on the collective signal response from all organisms in the sample, and hence are slow to detect any metabolic deviations. Although these methods are more rapid than conventional plating or turbidity methods, conventional impedance based detection methods and devices still require growth of the microbial organism to about 10⁷ CFU/ml in order to detect any metabolic deviations. The growth process can take up to 6 or more hours before there is sufficient growth to obtain results depending on the starting concentration of the organism. Moreover, the response from a conventional biological impedance device is very sensitive to variations in temperature. Conventional impedance devices and methods attempt to control the temperature of the biological sample to about ±0.1° C. by measuring the absolute temperature of the biological sample to minimize the effects of the temperature variations. However, such a temperature variation makes it difficult to monitor early cell growth and metabolic deviations by measuring the electrical properties of the biological sample suspension because thermal effects will dominate the biological signal of interest which prevents reducing the amount of time required to monitor the metabolic deviations.

A related biological impedance-type device is disclosed in U.S. Patent Application Publication No. 20030036054, assigned to Purdue Research Foundation, incorporated by reference herein. The Purdue device utilizes a micro-fabricated biochip with very small sample wells which typically hold about 100 nanoliters or less of sample fluid. Such a small sample volume is impractical for clinical applications. Moreover, the Purdue device is only used to monitor cellular growth and metabolic deviations of microorganisms and is not designed for sensitivity testing of the most effective antimicrobial drug for a microbial organism in a biological sample.

### BRIEF SUMMARY OF THE INVENTION

It is therefore an object of this invention to provide an improved system and method for rapid detection of metabolic deviations of a biological sample.

It is a further object of this invention to provide such an improved system and method which determines the sensitivity of a biological sample to a biological reagent in less time.

It is a further object of this invention to provide such an improved system and method which determines the most effective biological reagent for a biological sample in less time.

It is a further object of this invention to provide such an improved system and method which utilizes a larger sample volume.

It is a further object of this invention to provide such an improved system and method which is inexpensive.

It is a further object of this invention to provide such an improved system and method which is easy to operate.

It is a further object of this invention to provide such an improved system and method which can be used in a clinical environment.

It is a further object of this invention to provide such an improved system and method which determines sensitivity of a biological sample to a virus.

It is a further object of this invention to provide such an improved system and method which reduces the progression of the resistance of microbial organisms to antimicrobial drugs.

It is a further object of this invention to provide such an improved system and method which minimizes the effect of temperature on the measured metabolic deviations.

It is a further object of this invention to provide such an improved system which minimizes the effect of unwanted biological deviations in the measured metabolic deviations.

It is a further object of this invention to provide such an improved system which minimizes the effect of unwanted processes on the measured metabolic deviations.

This invention results from the realization that a truly innovative system and method for rapid detection of metabolic deviations of a biological sample can be achieved by measuring, at a first time, first impedances of a biological sample disposed in a control chamber and a biological sample and a biological reagent disposed in a target chamber, measuring, at a second time, second impedances of the biological sample in the control chamber and the biological reagent and the biological sample in the target chamber and then comparing the measured second impedances to the first measured impedances to measure if the biological reagent has induced any metabolic deviations to the biological sample in the target chamber and determine if the biological sample is sensitive to the biological reagent.

This invention features a system for rapid detection of metabolic deviations of a biological sample including at least one control chamber having spaced electrodes for receiving a biological sample, at least one target chamber having spaced electrodes for receiving the biological sample and a biological reagent, and a measuring and detection device responsive to the control chamber and the target chamber for measuring, at a first time, first impedances of the biological sample in the control chamber and the biological reagent and the biological sample in the target chamber and, at a second time, measuring second impedances of the biological sample in the control chamber and the biological sample and the biological reagent in the target chamber and comparing the second impedances to the first impedances to measure the effect of the biological reagent inducing metabolic deviations to the biological sample in the target chamber and determine whether the biological sample is sensitive to the biological reagent.

In one embodiment, the spaced electrodes of the control chamber may have an inner surface area and an outer surface area. The ratio of the inner surface area of one of the spaced electrodes of the control chamber to the distance between the spaced electrodes may optimize sensitivity of the system. The ratio may be greater than about 25. Each of the spaced electrodes of the target chamber may have an inner surface area and an outer surface area. The ratio of the inner surface area of one of the spaced electrodes of the target chamber to the distance between the spaced electrodes may optimize sensitivity of the system. The ratio may be greater than about 25. The measuring and detection device may measure, at the first time, a first plurality of impedances of the biological sample in the control chamber and the biological reagent and the biological sample in the target chamber and, at the second time, measure a second plurality of impedances of the biological sample in the control chamber and the biological reagent and the biological sample in the target chamber and compare the second plurality of impedances to the first plurality of impedances to measure the effect of the biological reagent inducing metabolic deviations to the biological sample in the target chamber and determine whether the biological sample is sensitive to the biological reagent. The biological sample may be chosen from the group consisting of urine, blood, sputum, feces, spinal fluid, food, water, and a solution containing a microbial organism. The biological sample may include one or more microbial organisms. The microbial organism may be chosen from the group consisting of Escherichia coli, Staphylococcus aureus, Enterococcus faecalis, Streptococcus pneumoniae, Neisseria gonorrhoeae, Mycobacterium tuberculosis, and Saccharomyces cerevisiae. The metabolic deviations may be chosen from the group consisting of morphological changes, changes in production of metabolites, surface charge changes, cell fission; cell growth, filamentation, enlargement or reduction in size of biological cells, metabolic end-products, and cell lysis. The lysis may be induced by a virus and/or a drug. The biological reagent may include any compound that induces a metabolic deviation and/or metabolic deviations to the biological sample. The biological reagent may be chosen from the group consisting of one or more anti-microbial compounds, antibiotics, viruses, enzymes, proteins, and molecular recognition probes. The absence of any measured metabolic deviations may indicate the biological sample is non-responsive and/or resistant to the biological reagent. The at least one control chamber and the at least one target chamber may have a volume in the range of about 0.01 ml to 1 ml. The value may be in the range of about 0.02 ml to 0.2 ml. The measuring and detection device may include a common mode rejection system for isolating the measured metabolic deviations induced by the biological reagent to the biological sample. The common mode rejection system may subtract the second measured impedances from the first measured impedances to provide a relative impedance indicative of the metabolic deviations. The common mode rejection system may minimize the unwanted effects of a temperature difference between the control chamber and the target chamber by differencing the temperature of the control chamber and the target chamber to provide a relative temperature difference between the control chamber and the target chamber. The relative temperature difference may be about 0.01° C. The relative temperature difference may be about 0.167° C. The common mode rejection system may minimize any unwanted biological growth deviations of the biological sample in the control chamber and in the target chamber by differencing the unwanted biological growth of the biological sample in the control chamber from the unwanted biological growth of the biological sample in the target chamber. The common mode rejection system may minimize any unwanted chemical deviations of the biological sample in the control chamber and in the target chamber by differencing the unwanted chemical deviations in the control chamber from the unwanted chemical deviations in the target chamber. The common mode rejection system may minimize any unwanted mechanical deviations by differencing the unwanted mechanical deviations in the control chamber and the target chamber. The biological sample may be disposed in a biological suspension. The biological sample in the at least one control chamber and the biological sample in the at least one target chamber may have an equal number of cells. The biological sample disposed in the at least one control chamber and the at least one target chamber may include identical strains of bacteria. The system may include a plurality of thermal platens disposed proximate to a top surface and a bottom surface of the control chamber and the target chamber for maintaining an approximately constant temperature of the control chamber and the target chamber. The approximately constant temperature may be in the range of about 20° C. to 50° C. The constant temperature may be about 37° C. The system may include a computer for executing software to evaluate the measured second impedances and the measured first impedances to indicate whether the biological reagent induced any metabolic deviations of the biological sample. The system may further include a plurality of target chambers and a control chamber connected to the measuring and detection device for measuring the effect of a plurality of biological reagents inducing metabolic deviations to the biological sample. The system may further include a plurality of reagent wells and a control well for holding a plurality of biological reagents and a control reagent. The system may further include a biological sample well for holding the biological sample. The system may further include a plurality of fluidic channels for interconnecting the plurality of target chambers, the control chamber, the plurality of reagent wells, the control well, and the biological sample well. The plurality of target chambers, the control chamber, the plurality of reagent wells, the control well, the sample well and the fluidic channels may be integrated into a cassette. The system may include a vacuum subsystem for driving the biological sample from the biological sample well into the plurality of reagent wells, the control well and into the plurality of target chambers and the control chamber via the fluidic channels. The system may include a hydrophobic filter disposed within one or more of the fluidic channels to contain the biological sample within the target chamber and the control chamber. The system may include top and bottom impedance guards disposed between the top surface and bottom surface of the control chamber and the target chamber and the plurality of thermal platens for shielding stray signals from the spaced electrodes of the control chamber and the target chamber to improve the first and second measured impedances. The common mode rejection system may be responsive to signals from the top and bottom impedance guards and the spaced electrodes of the control chamber and the target chamber for isolating and removing extraneous signals from the spaced electrodes of the control chamber and the target chamber. The system may determine that the biological sample is sensitive to the biological reagent in about 1 to 300 minutes for fast growing organisms. The system may determine that the biological sample is sensitive to the biological reagent in about 10 to 180 minutes for fast growing organisms. The system may determine that the biological sample is sensitive to the biological reagent in about 1 to 72 hours for slow growing organisms. The system may determine that the biological sample is sensitive to the biological reagent in about 2 to 12 hours for slow growing organisms.

This invention also features a system for rapid detection of metabolic deviations of a biological sample including at least one control chamber having spaced electrodes for receiving a biological sample, at least one target chamber having spaced electrodes for receiving the biological sample and a biological reagent, and a measuring and detection device responsive to the control chamber and the target chamber for measuring, at a first time, a first plurality of impedances of the biological sample in the control chamber and the biological reagent and the biological sample in the target chamber and, at a second time, measuring a second plurality of impedances of the biological sample in the control chamber and the biological sample and the biological reagent in the target chamber and comparing the second plurality of impedances to the first plurality of impedances to measure the effect of the biological reagent inducing metabolic deviations to the biological sample in the target chamber and determine whether the biological sample is sensitive to the biological reagent.

This invention also features a system for rapid detection of metabolic deviations of a biological sample including at least one control chamber having spaced electrodes for receiving a biological sample, the electrodes of the control chamber each having an inner and an outer surface area wherein the ratio of the inner surface area of one of the spaced electrodes to the distance between the spaced electrodes optimizes the sensitivity of the system, at least one target chamber having spaced electrodes for receiving the biological sample and a biological reagent, the electrodes of the target chamber each having an inner and an outer surface area wherein the ratio of the inner surface area of one of the spaced electrodes to the distance between the spaced electrodes optimizes the sensitivity of the system, and a measuring and detection device responsive to the control chamber and the target chamber for measuring, at a first time, first impedances of the biological sample in the control chamber and the biological reagent and the biological sample in the target chamber and, at a second time, measuring second impedances of the biological sample in the control chamber and the biological sample and the biological reagent in the target chamber and comparing the second impedances to the first impedances to measure the effect of the biological reagent inducing metabolic deviations to the biological sample in the target chamber and determine whether the biological sample is sensitive to the biological reagent.

In one embodiment, the ratio is greater than about 25.

This invention also features a system for rapid detection of metabolic deviations of a biological sample including at least one control chamber having spaced electrodes for receiving a biological sample, the electrodes of the control chamber each having an inner and an outer surface area wherein the ratio of the inner surface area of one of the spaced electrodes to the distance between the spaced electrodes optimizes the sensitivity of the system, at least one target chamber having spaced electrodes for receiving the biological sample and a biological reagent, the electrodes of the target chamber each having an inner and an outer surface area wherein the ratio of the inner surface area of one of the spaced electrodes to the distance between the spaced electrodes optimizes the sensitivity of the system, and a measuring and detection device responsive to the control chamber and the target chamber for measuring, at a first time, a first plurality of impedances of the biological sample in the control chamber and the biological reagent and the biological sample in the target chamber and, at a second time, measuring a second plurality of impedances of the biological sample in the control chamber and the biological sample and the biological reagent in the target chamber and comparing the second plurality of impedances to the first plurality of impedances to measure the effect of the biological reagent inducing metabolic deviations to the biological sample in the target chamber and determine whether the biological sample is sensitive to the biological reagent.

In one embodiment, the ratio is greater than about 25.

This invention further features a system for rapid detection of metabolic deviations of a biological sample including a cassette including a control chamber having spaced electrodes for receiving a biological sample and one or more target chambers having spaced electrodes for receiving the biological sample and a biological reagent, and a measuring and detection device for receiving the cassette and responsive to the control chamber and the target chamber for measuring, at a first time, first impedances of the biological sample in the control chamber and the biological reagent and the biological sample in the target chamber and, at a second time, measuring second impedances of the biological sample in the control chamber and the biological sample and the biological reagent in the target chamber, and comparing the second impedances to the first impedances to measure the effect of the biological reagent inducing metabolic deviations to the biological sample in the target chamber and determine whether the biological sample is responsive to the biological reagent.

This invention also features a system for rapid detection of metabolic deviations of a biological sample including a cassette including a sample well for receiving a biological sample, a control well for receiving a control reagent, one or more control chambers having spaced electrodes for receiving the biological sample, a plurality of reagent wells for receiving a plurality of biological reagents, and a plurality of target chambers each having spaced electrodes for receiving the biological sample and a biological reagent, and a measuring and detection device for receiving the cassette and responsive to the one or more control chambers and the plurality of target chambers for, at a first time, measuring first impedances of the biological sample in the one or more control chambers and the plurality of biological reagents and the biological sample in the plurality of target chambers and, at a second time, measuring second impedances of the biological sample in the one or more control chambers and the plurality of biological reagents and the biological sample in the plurality of target chambers and comparing the second impedances to the first impedances to measure the effect of the plurality of biological reagents inducing metabolic deviations to the biological sample in any of the plurality of target chambers and determine whether the biological sample is responsive to any of the plurality of biological reagents.

In one embodiment, the system may include fluidic channels for disposing the biological sample to the control well, the plurality of reagent wells, the one or more control chambers, and the plurality of target chambers.

This invention further features a method for rapid detection of metabolic deviations including measuring, at a first time, first impedances of a biological sample in a control chamber and a biological reagent and the biological sample in a target chamber, measuring, at a second time, second impedances of the biological sample in a control chamber and the biological reagent and the biological sample in the target chamber, and comparing the measured second impedances to the measured first impedances to measure the effect of the biological reagent inducing metabolic deviations to the biological sample in the target chamber and determine whether the biological sample is sensitive to the biological reagent.

This invention also features a method for rapid detection of metabolic deviations including measuring, at a first time, a first plurality of impedances of a biological sample in a control chamber and a biological reagent and the biological sample in a target chamber, measuring, at a second time, a second plurality of impedances of the biological sample in the control chamber and the biological reagent and the biological sample in the target chamber, and comparing the measured second plurality of impedances to the measured first plurality of impedances to measure the effect of the biological reagent inducing metabolic deviations to the biological sample in the control chamber and determine whether the biological sample is sensitive to the biological reagent.

### BRIEF DESCRIPTION OF THE DRAWINGS

Other objects, features and advantages will occur to those skilled in the art from the following description of a preferred embodiment and the accompanying drawings, in which:

FIG. 1 is a schematic block diagram of one embodiment of the system for rapid detection of metabolic deviations of a biological sample of this invention;

FIG. 2 is a schematic three-dimensional view showing in further detail the structure of the spaced electrodes shown in FIG. 1;

FIG. 3 is a schematic top view of another embodiment of the system for rapid detection of metabolic deviations of a biological sample employing a plurality of target chambers and a control chamber in accordance with this invention;

FIG. 4 is a schematic three-dimensional view taken along line 3-3 of FIG. 3 showing one embodiment of a removable cassette used for sample collection of and testing of the sensitivity of the sample to a plurality of biological reagents in accordance with this invention;

FIG. 5 is a three-dimensional view showing the removable cassette of FIG. 3 in use with the system for rapid detection of metabolic deviations of this invention;

FIG. 6 is a schematic side view of another embodiment of the system for rapid detection of metabolic deviations of this invention employing thermal platens and capacitive guards;

FIG. 7 is a graph showing an example of the rapid detection of metabolic deviations which indicate a biological sample is sensitive to various biological reagents in accordance with this invention; and

FIG. 8 is a block diagram showing the primary steps for rapid detection of metabolic deviations in accordance with this invention.

### PREFERRED EMBODIMENT

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

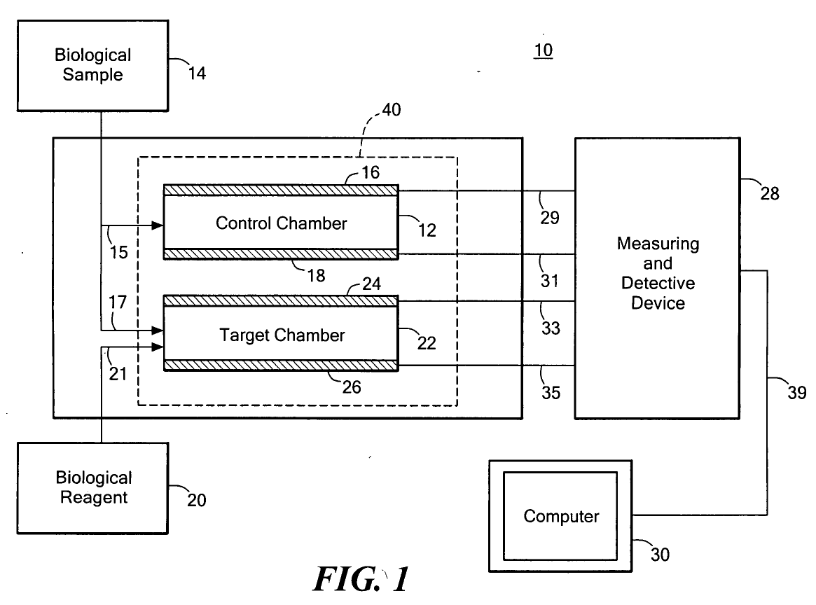

There is shown in FIG. 1, system 10 for rapid detection of metabolic deviations of a biological sample of this invention. System 10 includes control chamber 12 which receives biological sample 14 via fluidic channel 15. Control chamber 12 also includes spaced electrodes 16 and 18 which are used to measure the impedance of biological sample 14 disposed in control chamber 12, as discussed in further detail below. Biological sample 14 may be blood, urine, sputum, feces, spinal fluid, food, water, a solution containing one or more microbial organisms, or any other biological sample known to those skilled in the art. The microbial organisms in biological sample 14 may include any biological cell, such as Escherichia coli, Staphylococcus aureus, Enterococcus faecalis, Streptococcus pneumoniae, Neisseria gonorrhoeae, Mycobacterium tuberculosis, Saccharomyces cerevisiae, or any other microbial organism known to those skilled in the art. Target chamber 22 receives biological sample 14 via fluidic channel 17 and biological reagent 20 via fluidic channel 21. Biological reagent 20 may include one or more antimicrobial compounds, antibiotics, viruses, enzymes, proteins, molecular recognition probes, or any other known biological reagent or reagents known to those skilled in the art. Target chamber 22 includes spaced electrodes 24 and 26 which measure the impedance of biological sample 14 and biological reagent 20 in target chamber 22, as discussed in further detail below. Control chamber 12 and target chamber 22 are typically mechanically identical and in close proximity to one another to promote the common mode rejection of unwanted effects, also discussed in detail below. Typically, a growth media, such as tryptic soy broth (TSB) is disposed in both control chamber 12 and target chamber 22 to promote the growth of any microbial organisms that may be present in biological sample 14.

System 10 includes measuring and detection device 28 connected to electrodes 16 and 18 of control chamber 12 by lines 29 and 31 and electrodes 24 and 26 of target chamber 22 by lines 33 and 35. Measuring and detection device 28 may be an AT-4284A precision LCR meter, available from Agilent Technologies (Palo Alto, Calif.). Measuring and detection device 28 measures, at a first time, a first impedance of biological sample 14 in control chamber 12 and a first impedance of biological sample 14 and biological reagent 20 in target chamber 22. Then, at a second time, e.g., about 10 to 180 minutes later for fast growing organisms, or about 2 to 12 hours for slow growing organisms, measuring and detection device 28 measures a second impedance of biological sample 14 in control chamber 12 and a second impedance of biological sample 14 and biological reagent 20 in target chamber 22. Measuring and detection device 28 then compares the measured second impedances to the measured first impedances to determine if biological reagent 20 has induced any metabolic deviations to biological sample 14 in target chamber 22. Typically, system 10 can measure and detect metabolic deviations induced by biological reagent 20 on biological sample 14 in control chamber 12 in about 1 to 300 minutes for fast growing organisms with a typical detection time of about 10 to 180 minutes. Similarly, system 10 can measure and detect metabolic deviations induced by biological reagent 20 on biological sample 14 in control chamber 12 in about 1 to 72 hours for slow growing organisms, with a typical detection time of about 2 to 12 hours.

Metabolic deviations which may be induced to biological sample 14 by biological reagent 20 include, inter alia, morphological changes, changes in production of metabolites, surface charge changes, cell fission, cell growth, filamentation, enlargement or reduction in size of biological cells, metabolic end-products, cell lysis induced by a drug, a virus, e.g., the infection of Bacillus subtilis by phase SP01, or the bactericidal actions of ampicillin on E. coli, or any other metabolic deviations known by those skilled in the art. If the second measured impedance (e.g., 26.190 Ω) of biological sample 14 and biological reagent 20 in target chamber 22 is different than the first measured impedance (e.g., 26.096 Ω) of biological sample 14 and biological reagent 20 in target chamber 22 while the second and first measured impedances (e.g., 25.715 Ω and 25.720 Ω) of the biological sample 14 in control chamber 12 remain relatively constant, then biological reagent 20 has induced metabolic deviations to biological sample 14 which indicates biological sample 14 in target chamber 22 is sensitive to biological reagent 20. If the second and first measured impedances of biological sample 14 and biological reagent 20 in target chamber 22 are the same, then biological reagent 20 has not induced any metabolic deviations to biological sample 14 which indicates biological sample 14 is insensitive to biological reagent 20.

In one embodiment, measuring and detection device 28 measures, at a first time, a first plurality of impedances of biological sample 14 in control chamber 12 and a first plurality of impedances of biological sample 14 and biological reagent 20 in target chamber 22. Similarly as described above, at a second time, measuring and detection device 28 measures a second plurality of impedances of biological sample 14 in control chamber 12 and a second plurality of impedances of biological sample 14 and biological reagent 20 in target chamber 22. Measuring and detection device 28 then compares the measured second plurality of impedances to the measured first plurality of impedances to determine if biological reagent 20 has induced any metabolic deviations to biological sample 14 in target chamber 22. Measuring a first plurality and second plurality of impedances increases the number of data points output by line 39, which provides a more accurate signature of any metabolic deviations induced by biological reagent 20 on biological sample 14 in target chamber 22 which are input to computer 30.

Computer 30, e.g., a personal computer, is responsive to output signals (data) of measuring and detection device 28 on line 39 and typically includes software and algorithms which evaluate the output signals on line 39 to designate the tested biological sample 14 as sensitive to biological reagent 20.

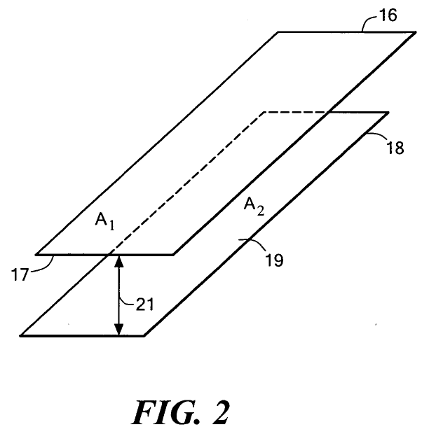

Spaced electrodes 16 and 18 of control chamber 12 and spaced electrodes 24 and 26 of target chamber 22 typically have equal surface areas and are designed such that the ratio of the surface area of any one of electrodes 16 and 18 or electrodes 24 and 26 and the gap distance between them is greater than about 25 to improve the sensitivity of system 10. For example, FIG. 2 shows spaced electrodes 16 and 18 of control chamber 12, FIG. 1. In this example, inner surface area 17 (A₁), FIG. 2, of electrode 16 is equal to inner surface area 19 (A₂) of electrode 18. In this design, the ratio of surface area 17 (A₁) or surface area 19 (A₂) to the gap distance between electrodes 16 and 18, indicated by arrow 21, is greater than about 25 which improves the sensitivity of system 10. Electrodes 24 and 26 of target chamber 22 have a similar design as described above.

The result is that system 10, FIG. 1, of this invention can rapidly determine the sensitivity of a biological sample to a biological reagent in as little as about 15 to 30 minutes for fast growing organisms (or 2 to 12 hours for slow growing organisms), which is a significant improvement over conventional sensitivity testing methods which typically take 12 to 24 hours or more to complete (or days for slow growing organisms). Hence, physicians can quickly prescribe the most effective biological reagent (e.g., an antibiotic) for a particular microbial organism causing an illness or disease, which provides more effective treatment to the patient and reduces the progression of microorganisms becoming resistant to antimicrobial drugs. System 10 is also easy to operate, as discussed in further detail below, and hence, can be used in a clinical environment which eliminates the need to send the biological sample to the laboratory to determine the sensitivity of the biological sample to the biological reagent, which saves time and decreases costs. Moreover, system 10 can quickly and easily determine the sensitivity of the biological sample 14 to viruses and bacteriophages, as discussed in further detail below.

Control chamber 12 and target chamber 22 typically have volumes in the range of about 0.1 ml to 1 ml. In one design, the volume of control chamber 12 and target chamber 22 is about 0.02 to about 0.25 ml. The combination of the reduced volume and the ratio of the area of electrodes 16, 18, 24, and 26 to the distance between electrodes 16 and 18 or electrodes 24 and 26 as discussed above provides for rapid detection of metabolic deviations and the ability to rapidly determine whether biological sample 14 is sensitive to biological reagent 20. In one design this ratio is equal to 1916. The increased volume of target chamber 22 and control chamber 12 is practical for clinical applications, e.g., a physician's office. As discussed below, system 10 may include a separate biological sample well which also has a volume size up to 1 ml and receives biological samples which may be dispensed with a pipette or syringe.

System 10 includes common mode rejection system 40 which isolates the measured effect of biological reagent 20 inducing metabolic deviations to biological sample 14 in target chamber 22. The measured impedance signals from chambers 12 and 22 typically include the effects from environmental, chemical, biological phenomena, and the like. Because control chamber 12 and target chamber 22 are ideally mechanically identical chambers containing biological sample 14 in close proximity to each other, the respective impedance signals differ by only the effects from the introduction of biological reagent 20 to biological sample 14. Common mode rejection techniques known to those skilled in the art are performed with common mode rejection system 40. For example, common mode rejection system 40 may subtract the second measured impedances of biological sample 14 in control chamber 12 and biological reagent 20 and biological sample 14 in target chamber 22 from the first measured impedances of biological sample 14 in control chamber 12 and biological reagent 20 and biological sample 14 in target chamber 22 to provide relative impedances which are indicative of the metabolic deviations induced (if any) by biological reagent 20 to biological sample 14 in target chamber 22. Common mode rejection system 40 may also minimize the effects on the impedance signal response from temperature on biological sample 14 in target chamber 22 by differencing the impedance response of biological sample 14 in control chamber 12 and target chamber 22 to provide a relative response relying on the effects from the temperature difference between the two chambers, e.g., non-absolute, between control chamber 12 and target chamber 22. The result is that system 10 no longer relies on the effects from the absolute temperature of the biological sample, as found in the prior art, and instead measures and controls effects from the relative temperature of the biological sample 14 in control chamber 12 and target chamber 22. In one example, common mode rejection system 40 utilizes the relative temperature, e.g., non-absolute temperature of biological sample 14 in control chamber 12 and biological sample 14 and biological reagent 20 in target chamber 22 at a temperature of about 0.01° C. or better. In one preferred example, the relative temperature utilized is about 0.0167° C. while the absolute temperature is controlled to only 2.3° C. Minimizing the effects from temperature on biological sample 14 in control chamber 12 and biological sample 14 and biological reagent 20 in target chamber 22 provides the ability for system 10 to monitor metabolic deviations of biological sample 14 more accurately and faster because the effects of thermal variations do not dominate the measured biological response.

Common mode rejection system 40 may also minimize unwanted metabolic growth deviations of biological sample 14 in control chamber 12 and target chamber 22 by differencing the biological growth (if any) of biological sample 14 in control chamber 12 from the biological growth of biological sample 14 in target chamber 22. Common mode rejection system 40 may also minimize unwanted mechanical deviations by differencing any mechanical deviations, e.g., vibrations and the like, introduced to control chamber 12 and target chamber 22, as known by those skilled in the art. Common mode rejection system 40 may also minimize unwanted chemical deviations by differencing any chemical deviations, e.g., surface binding onto electrodes 16, 18, 24, and 26 or chemical degradation of biological sample 14, and the like, introduced to control chamber 12 and target chamber 22, as known by those skilled in the art.

Biological sample 14 is typically suspended in a biological suspension, e.g., E. coli in TSB nutrient broth before being disposed into control chamber 12 and target chamber 22. In one example, biological sample 14 in control chamber 12 and target chamber 22 have an equal number of cells. Biological sample 14 in target chamber 22 and control chamber 12 may have identical strains of bacteria, such as Escherichia coli, Staphylococcus aureus, Enterococcus faecalis, Streptococcus pneumoniae, Neisseria gonorrhoeae, Mycobacterium tuberculosis, Saccharomyces cerevisiae, or any other microbial organism known to those skilled in the art. In other examples, cell lysis of any microbial organisms in biological sample 14 may be caused by a biological reagent 20 which contains one or more viruses and/or bacteriophages which induce metabolic deviations by causing the lysis of any microbial organisms in biological sample 14. The metabolic deviations caused by the cell lysis are measured by measuring and detection device 28, and similarly as described above, rapidly indicate sensitivity of biological sample 14 to one or more viruses and/or bacteriophages. In other examples, biological reagent 20 may include a drug, e.g., ampicillin, which induces the cell lysis.

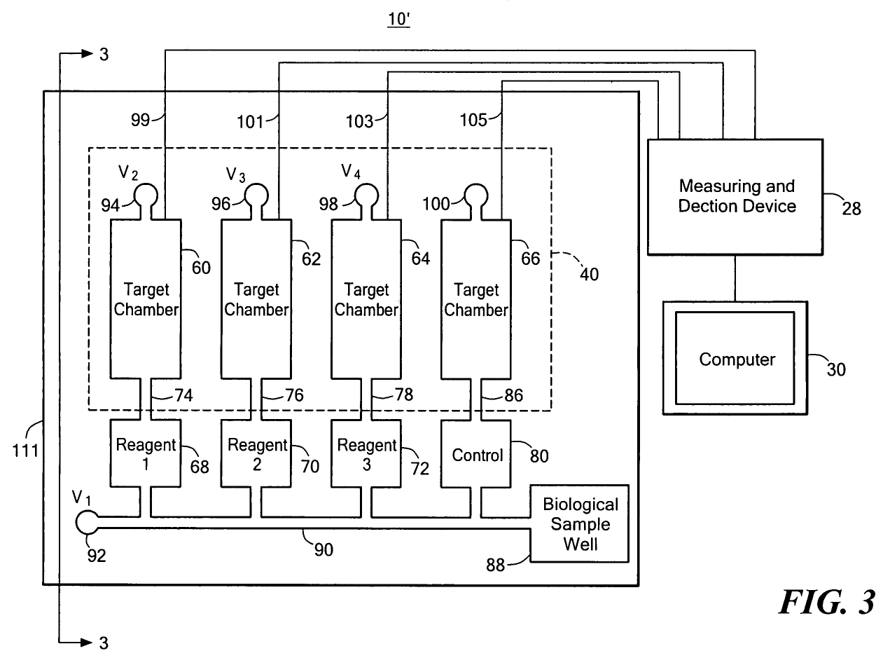

Another system for rapid detection of metabolic deviations 10′, FIG. 3, where like parts have been given like numbers, includes a plurality of target chambers, such as target chambers 60, 62 and 64 and control chamber 66. System 10′ also includes a plurality of reagent wells, such as reagent wells 68, 70, and 72 which hold a plurality of biological reagents. Reagent wells 68, 70 and 72 are interconnected to target chambers 60, 62, and 64 via fluidic channels 74, 76, and 78, respectively. System 10′ also includes a control well 80 which holds a control reagent, e.g., a non-reactive solution, such as a growth media, and no biological reagent. Reagent wells 68-72 and control well 80 also typically contain a growth media, such as TSB, described above. Control well 80 is interconnected to control chamber 66 via fluidic channel 86. System 10′ also includes biological sample well 88 for holding a biological sample and typically has a volume of about 0.01 ml to 1 ml, which is practical for clinical and other applications. The biological sample in sample well 88 is introduced and mixed with the biological reagents in reagent wells 68-72 and the control well 80 via fluidic channel 90. Vacuum ports 92, 94, 96, 98, and 100 are connected to a vacuum subsystem (not shown) and drive the biological sample in sample well 88 via fluidic channel 90 to reagent wells 68-72 and control well 80. The mixed solution of the biological reagents and the biological sample in reagent wells 68-72 and control solution in control well 80 are driven to target chambers 60-64 and control chamber 66 via fluidic channels 74-78 and 86, respectively. Target chambers 60-64, control chamber 66, reagent wells 68-72, control well 80, sample well 88 and fluidic channels 74, 76, 78, 86 and 90 are typically integrated onto substrate 111, e.g., a plastic material or similar material known to those skilled in the art. As discussed below, substrate 111 may define a removable cassette which is practical for clinical applications. Target chambers 60-64 and control chamber 66 have spaced electrodes of similar design as electrodes of the target chamber and the control chamber as described above in reference to FIG. 1. System 10′, FIG. 3 includes measuring and detection device 28 connected to target chambers 60-64 and control chamber 66 by lines 99, 101, 103, and 105, respectively, and measures metabolic deviations (if any) of the biological sample in target chambers 60-64 which may be induced by a plurality of biological reagents in reagent wells 68-72 to determine the sensitivity (or lack thereof) of the biological sample to the plurality of biological reagents.

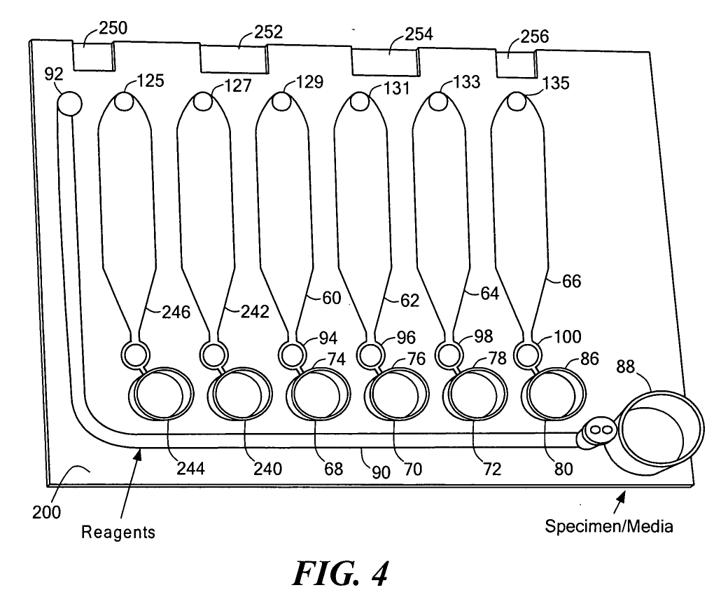

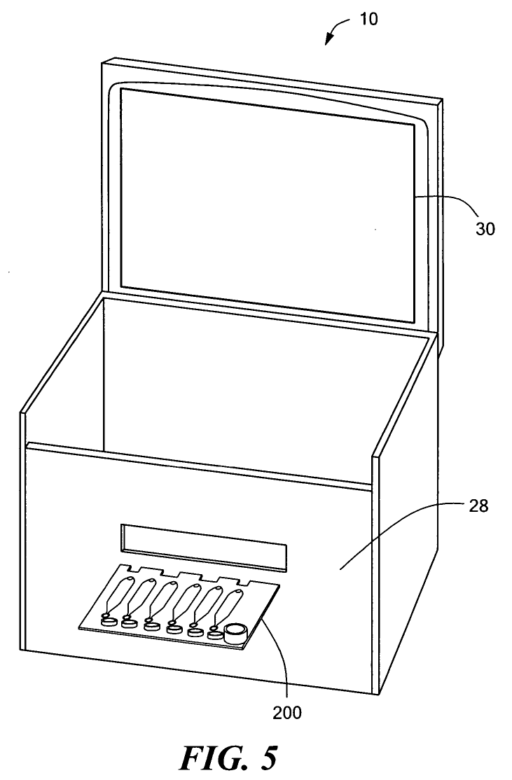

The plurality of target chambers 60-64, control chamber 66, sample well 88, reagent wells 68-72, control well 80, fluidic channels 74, 76, 78, 86, and 90 and vacuum ports 92-100, 125, 127, 129, 131, 133 and 135 may be integrated into removable cassette 200, FIG. 4, where like parts have been given like numbers. pCassette 200 provides for simplified and easy use in a clinical environment. Cassette 200 is typically composed of plastic or similar material known to those skilled in the art. Biological sample well 88 typically has a volume of as large as 1 ml and can receive a biological sample via a syringe, pipette, or similar dispensing device which can be dispensed by non-laboratory-skilled personnel. Cassette 200 may also include additional reagent well 240, target chamber 242, control well 244, and control chamber 246 which provide for additional biological reagents and control reagents. Cassette 200 includes electric pads 250, 252, 254, and 256 which provide an interconnection to measuring and detection device 28, FIG. 5. As described above, measuring and detection device 28 determines the sensitivity of the biological sample in well 88, FIG. 4, to the plurality of biological reagents in reagent wells 68-72. Similarly, computer 30 provides an indication of the sensitivity of the biological sample to the various biological reagents which have reacted (or not reacted) with the biological sample and induced (or not induced) metabolic deviations to determine whether the biological sample is sensitive to the various biological reagents. Cassette 200 is typically 5 inches in length, 3 inches wide, and 0.5 inches thick. Because cassette 200 has a large sample well size and an overall size which is practical for clinical applications, biological samples can easily be disposed in sample well 88 with a pipette, syringe or similar device. Removable cassette 200 can easily be transported and inserted into measuring and detection device 28, FIG. 5. The result is that system 10 is easy to operate, eliminates the need to send the sample to a laboratory, and reduces the time to determine the sensitivity of a biological sample to one or more biological reagents.

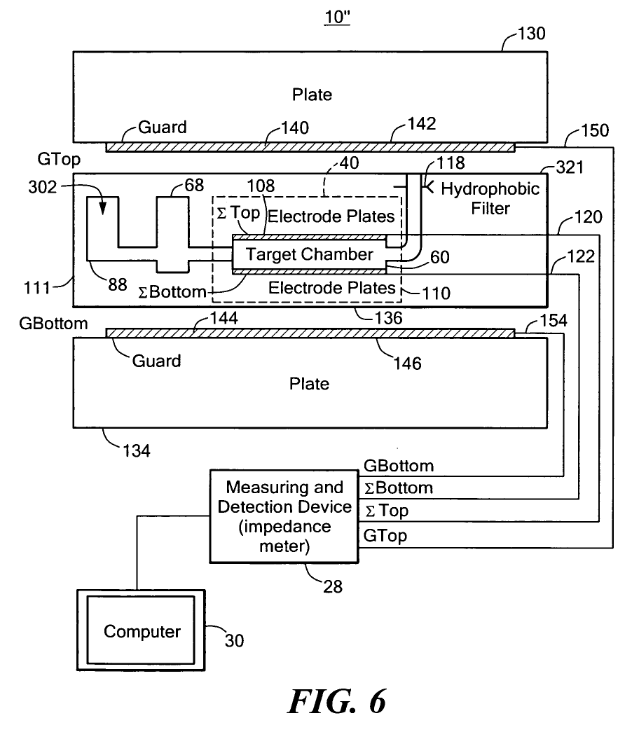

System 10″, FIG. 6 of this invention, taken along line 3-3 of FIG. 3, where like parts have been given like numbers, includes sample well 88 which receives a biological sample as indicated by arrow 302. Reagent well 68 contains the biological reagent which is mixed with the biological sample in target chamber 60. Target chamber 60 (as well as target chambers 62 and 64, and control chamber 66, as shown in FIG. 3) includes top electrode 108 and bottom electrode 110. Leads 120 and 122 connect top electrode 108 and bottom electrode 110 to measuring and detection device 28. Top platen 130 is disposed proximate to top surface 132 of substrate 111 and platen 134 is disposed proximate to bottom surface 136 of substrate 111. Platens 130 and 134 maintain approximately constant temperature of target chamber 60 (as well as target chambers 62 and 64 and control chamber 66, FIG. 3) typically in the range of about 20° C. to 50° C. In a preferred embodiment, the constant temperature of target chamber 60, FIG. 6, (as well as target chambers 62 and 64, FIG. 3, and control chamber 66) is about 37° C. Hydrophobic filter 118, FIG. 6, contains the biological sample and the biological reagent within target chamber 60.

System 10″ also includes impedance guard 140 disposed on bottom surface 142 of platen 130 and impedance guard 144 disposed on top surface 146 of platen 134. Impedance guards 140 and 144 shield stray signals from top electrode 108 and bottom electrode 110 and improve the measured first and second impedances measured by measuring and detection device 28. Leads 150 and 154 connect impedance guards 140 and 144 to measuring and detection device 28, respectively. Platens 130 and 134 typically span target chambers 62 and 64, FIG. 3, and control chamber 66.

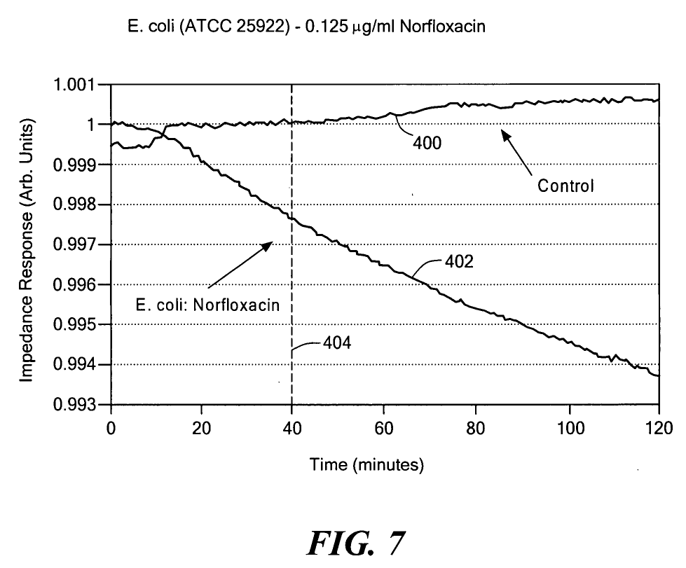

FIG. 7 shows examples of the rapid detection of metabolic deviations of a biological sample to the biological reagent Norfloxacin in accordance with this invention. In this example, curve 400, shows the measured response of a control biological sample containing E. coli. Curve 402 shows the measured response of a biological sample containing E. coli which is sensitive to the biological reagent Norfloxacin. As shown by dashed line 404, the sensitivity of the biological sample to the biological reagent Norfloxacin is detected by the system of this invention in about 40 minutes.

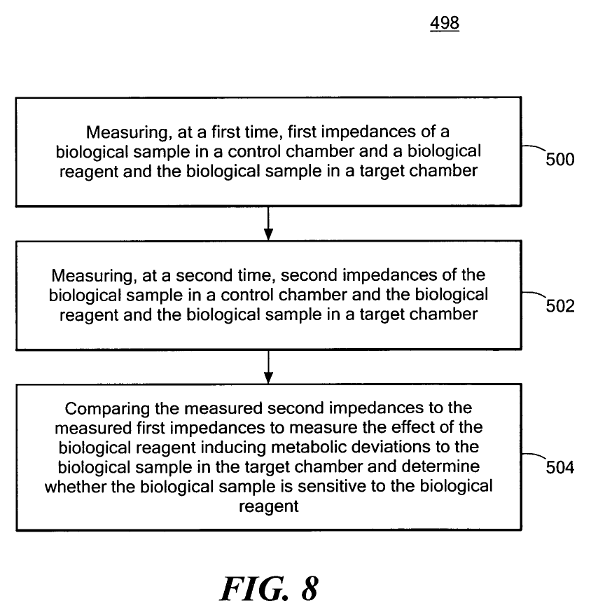

The method for rapid detection of metabolic deviations 498, FIG. 8, of this invention includes the steps of measuring at a first time, first impedances of a biological sample in a control chamber and a biological reagent and the biological sample in a target chamber, step 500, measuring, at a second time, second impedances of the biological sample in the control chamber and the biological sample and the biological reagent in the target chamber, step 502, and comparing the measured second impedances to the measured first impedances to determine whether the biological reagent has induced any metabolic deviations in the biological sample and determine whether the biological sample is sensitive to the biological reagent, step 504.

The method as described above may also measure, at the first time, a first plurality of impedances of the biological sample in the control chamber and the biological sample and the biological reagent in the target chamber and measure, at the second time, a second plurality of impedances of the biological sample in the control chamber and the biological sample and the biological reagent in the target chamber and compare the measured second plurality of impedances to the measured first plurality of impedances to determine if the biological reagent has induced any metabolic deviations in the biological sample to determine if the biological sample is sensitive to the biological reagent.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

1. A system for rapid detection of metabolic deviations of a biological sample comprising:
at least one control chamber having spaced electrodes for receiving a biological sample;
at least one target chamber having spaced electrodes for receiving the biological sample and a biological reagent; and
a measuring and detection device responsive to said control chamber and said target chamber for measuring, at a first time, first impedances of said biological sample in said control chamber and the biological reagent and the biological sample in said target chamber and, at a second time, measuring second impedances of the biological sample in said control chamber and the biological sample and the biological reagent in said target chamber and comparing said second impedances to said first impedances to measure the effect of the biological reagent inducing metabolic deviations to the biological sample in said target chamber and determine whether the biological sample is sensitive to the biological reagent.

2. The system of claim 1 in which each of said spaced electrodes of said control chamber have an inner surface area and an outer surface area.

3. The system of claim 2 in which the ratio of said inner surface area of one of said spaced electrodes of said control chamber to the distance between said spaced electrodes optimizes sensitivity of said system.

4. The system of claim 3 in which said ratio is greater than about 25.

5. The system of claim 1 in which each of said spaced electrodes of said target chamber have an inner surface area and an outer surface area.

6. The system of claim 5 in which the ratio of said inner surface area of one of said spaced electrodes of said target chamber to the distance between said spaced electrodes optimizes sensitivity of said system.

7. The system of claim 6 in which said ratio is greater than about 25.

8. The system of claim 1 in which said measuring and detection device measures, at said first time, a first plurality of impedances of said biological sample in said control chamber and the biological reagent and the biological sample in said target chamber and, at said second time, measures a second plurality of impedances of the biological sample in said control chamber and the biological reagent and the biological sample in said target chamber and compares said second plurality of impedances to said first plurality of impedances to measure the effect of the biological reagent inducing metabolic deviations to the biological sample in said target chamber and determine whether the biological sample is sensitive to the biological reagent.

9. The system of claim 1 in which said biological sample is chosen from the group consisting of: urine, blood, sputum, feces, spinal fluid, food, water, and a solution containing a microbial organism.

10. The system of claim 9 in which said biological sample includes one or more microbial organisms.

11. The system of claim 10 in which said microbial organism is chosen from the group consisting of: Escherichia coli, Staphylococcus aureus, Enterococcus faecalis, Streptococcus pneumoniae, Neisseria gonorrhoeae, Mycobacterium tuberculosis, and Saccharomyces cerevisiae.

12. The system of claim 1 in which said metabolic deviations are chosen from the group consisting of: morphological changes, changes in production of metabolites, surface charge changes, cell fission, cell growth, filamentation, enlargement or reduction in size of biological cells, metabolic end-products, and cell lysis.

13. The system of claim 12 in which said lysis is induced by a virus.

14. The system of claim 12 in which said lysis is caused by a drug.

15. The system of claim 1 in which said biological reagent includes any compound that induces a metabolic deviation and/or metabolic deviations to said biological sample.

16. The system of claim 1 in which said biological reagent is chosen from the group consisting of: one or more anti-microbial compounds, antibiotics, viruses, enzymes, proteins, and molecular recognition probes.

17. The system of claim 1 in which the absence of any measured metabolic deviations indicates said biological sample is non-responsive and/or resistant to said biological reagent.

18. The system of claim 1 in which said at least one control chamber and said at least one target chamber have a volume in the range of about 0.01 ml to 1 ml.

19. The system of claim 18 in which said at least one control chamber and said at least one target chamber have a volume in the range of about 0.02 ml to 0.2 ml.

20. The system of claim 1 in which said measuring and detection device includes a common mode rejection system for isolating the measured metabolic deviations induced by said biological reagent to said biological sample.

21. The system of claim 20 in which said common mode rejection system subtracts said second measured impedances from said first measured impedances to provide a relative impedance indicative of said metabolic deviations.

22. The system of claim 20 in which said common mode rejection system minimizes the effects of a temperature difference between said control chamber and said target chamber by differencing the temperature of said control chamber and said target chamber to provide a relative temperature difference between said control chamber and said target chamber.

23. The system of claim 22 in which said relative temperature difference is about 0.01° C.

24. The system of claim 22 in which said relative temperature difference is about 0.167° C.

25. The system of claim 20 in which said common mode rejection system minimizes any unwanted biological growth deviations of said biological sample in said control chamber and said target chamber by differencing said biological growth of said biological sample in said control chamber from said biological growth of said biological sample in said target chamber.

26. The system of claim 20 in which said common mode rejection system minimizes any unwanted chemical deviations of said biological sample in said control chamber and said target chamber by differencing said unwanted chemical deviations in said control chamber from said unwanted chemical deviations of in said target chamber.

27. The system of claim 20 in which said common mode rejection system minimizes any unwanted mechanical deviations by differencing said unwanted mechanical deviations in said control chamber and said target chamber.

28. The system of claim 1 in which said biological sample is disposed in a biological suspension.

29. The system of claim 1 in which said biological sample in said at least one control chamber and said biological sample in said at least one target chamber have an equal number of cells.

30. The system of claim 1 in which the biological sample disposed in said at least one control chamber and said at least one target chamber include identical strains of bacteria.

31. The system of claim 20 further including a plurality of thermal platens disposed proximate to a top surface and a bottom surface of said control chamber and said target chamber for maintaining an approximately constant temperature of said control chamber and said target chamber.

32. The system of claim 31 in which said approximately constant temperature is in the range of about 20° C. to 50° C.

33. The system of claim 32 in which said constant temperature is about 37° C.

34. The system of claim 1 further including a computer for executing software to evaluate said measured second impedances and said measured first impedances to indicate whether said biological reagent induced any metabolic deviations of said biological sample.

35. The system of claim 1 further including a plurality of target chambers and a control chamber connected to said measuring and detection device for measuring the effect of a plurality of biological reagents inducing metabolic deviations to the biological sample.

36. The system of claim 35 further including a plurality of reagent wells and a control well for holding a plurality of biological reagents and a control reagent.

37. The system of claim 36 further including a biological sample well for holding said biological sample.

38. The system of claim 37 further including a plurality of fluidic channels for interconnecting said plurality of target chambers, said control chamber, said plurality of reagent wells, said control well, and said biological sample well.

39. The system of claim 38 wherein said plurality of target chambers, said control chamber, said plurality of reagent wells, said control well, said sample well and said fluidic channels are integrated into a cassette.

40. The system of claim 38 further including a vacuum subsystem for driving said biological sample from said biological sample well into said plurality of reagent wells, said control well and into said plurality of target chambers and said control chamber via said fluidic channels.

41. The system of claim 38 further including a hydrophobic filter disposed within one or more of said fluidic channels to contain said biological sample within said target chamber and said control chamber.

42. The system of claim 31 further including top and bottom impedance guards disposed between said top surface and said bottom surface of said control chamber and said target chamber and said plurality of thermal platens for shielding stray signals from said spaced electrodes of said control chamber and said target chamber to improve said first and second measured impedances.

43. The system of claim 42 in which said common mode rejection system is responsive to signals from said top and bottom impedance guards and said spaced electrodes of said control chamber and said target chamber for isolating and removing extraneous signals from said spaced electrodes of said control chamber and said target chamber.

44. The system of claim 1 in which said system determines that said biological sample is sensitive to said biological reagent in about 1 to 300 minutes for fast growing organisms.

45. The system of claim 44 in which said system determines that said biological sample is sensitive to said biological reagent in about 10 to 180 minutes for fast growing organisms.

46. The system of claim 1 in which said system determines that said biological sample is sensitive to said biological reagent in about 1 to 72 hours for slow growing organisms.

47. The system of claim 46 in which said system determines that said biological sample is sensitive to said biological reagent in about 2 to 12 hours for slow growing organisms.

48. A system for rapid detection of metabolic deviations of a biological sample comprising:
at least one control chamber having spaced electrodes for receiving a biological sample;
at least one target chamber having spaced electrodes for receiving the biological sample and a biological reagent; and
a measuring and detection device responsive to said control chamber and said target chamber for measuring, at a first time, a first plurality of impedances of said biological sample in said control chamber and the biological reagent and the biological sample in said target chamber and, at a second time, measuring a second plurality of impedances of the biological sample in said control chamber and the biological sample and the biological reagent in said target chamber and comparing said second plurality of impedances to said first plurality of impedances to measure the effect of the biological reagent inducing metabolic deviations to the biological sample in said target chamber and determine whether the biological sample is sensitive to the biological reagent.

49. A system for rapid detection of metabolic deviations of a biological sample comprising:
at least one control chamber having spaced electrodes for receiving a biological sample, said electrodes each having an inner and an outer surface area wherein the ratio of said inner surface area of one of said spaced electrodes to the distance between said spaced electrodes optimizes the sensitivity of said system;
at least one target chamber having spaced electrodes for receiving the biological sample and a biological reagent, said electrodes each having an inner and an outer surface area wherein the ratio of said inner surface area of one of said spaced electrodes to the distance between said spaced electrodes optimizes the sensitivity of said system; and
a measuring and detection device responsive to said control chamber and said target chamber for measuring, at a first time, first impedances of said biological sample in said control chamber and the biological reagent and the biological sample in said target chamber and, at a second time, measuring second impedances of the biological sample in said control chamber and the biological sample and the biological reagent in said target chamber and comparing said second impedances to said first impedances to measure the effect of the biological reagent inducing metabolic deviations to the biological sample in said target chamber and determine whether the biological sample is sensitive to the biological reagent.

50. The system of claim 49 in which said ratio is greater than about 25.

51. A system for rapid detection of metabolic deviations of a biological sample comprising:
at least one control chamber having spaced electrodes for receiving a biological sample, said electrodes each having an inner and an outer surface area wherein the ratio of said inner surface area of one of said spaced electrodes to the distance between said spaced electrodes optimizes the sensitivity of said system;
at least one target chamber having spaced electrodes for receiving the biological sample and a biological reagent each having an inner and an outer surface area wherein the ratio of said inner surface area of one of said spaced electrodes to the distance between said spaced electrodes optimizes the sensitivity of said system; and
a measuring and detection device responsive to said control chamber and said target chamber for measuring, at a first time, a first plurality of impedances of said biological sample in said control chamber and the biological reagent and the biological sample in said target chamber and, at a second time, measuring a second plurality of impedances of the biological sample in said control chamber and the biological sample and the biological reagent in said target chamber and comparing said second plurality of impedances to said first plurality of impedances to measure the effect of the biological reagent inducing metabolic deviations to the biological sample in said target chamber and determine whether the biological sample is sensitive to the biological reagent.

52. The system of claim 51 in which said ratio is greater than about 25.

53. A system for rapid detection of metabolic deviations of a biological sample comprising:
a cassette including:
a control chamber having spaced electrodes for receiving a biological sample, and
one or more target chambers having spaced electrodes for receiving the biological sample and a biological reagent; and
a measuring and detection device for receiving said cassette and responsive to said control chamber and said target chamber for measuring, at a first time, first impedances of said biological sample in said control chamber and the biological reagent and the biological sample in said target chamber and, at a second time, measuring second impedances of the biological sample in said control chamber and the biological sample and the biological reagent in said target chamber, and comparing said second impedances to said first impedances to measure the effect of the biological reagent inducing metabolic deviations to the biological sample in said target chamber and determine whether the biological sample is responsive to the biological reagent.

54. A system for rapid detection of metabolic deviations of a biological sample comprising:
a cassette including:
a sample well for receiving a biological sample,
a control well for receiving a control reagent,
one or more control chambers having spaced electrodes for receiving the biological sample,
a plurality of reagent wells for receiving a plurality of biological reagents, and
a plurality of target chambers each having spaced electrodes for receiving the biological sample and a biological reagent; and
a measuring and detection device for receiving said cassette and responsive to said one or more control chambers and said plurality of target chambers for, at a first time, measuring first impedances of the biological sample in said one or more control chambers and said plurality of biological reagents and the biological sample in said plurality of target chambers and, at a second time, measuring second impedances of the biological sample in said one or more control chambers and said plurality of biological reagents and the biological sample in said plurality of target chambers and comparing said second impedances to said first impedances to measure the effect of said plurality of biological reagents inducing metabolic deviations to the biological sample in any of said plurality of target chambers and determine whether the biological sample is responsive to any of the plurality of biological reagents.

55. The system of claim 54 further including fluidic channels for disposing said biological sample to said control well, said plurality of reagent wells, said one or more control chambers, and said plurality of target chambers.

56. A method for rapid detection of metabolic deviations comprising:
measuring, at a first time, first impedances of a biological sample in a control chamber and a biological reagent and the biological sample in a target chamber;
measuring, at a second time, second impedances of the biological sample in a control chamber and the biological reagent and the biological sample in a target chamber; and
comparing the measured second impedances to the measured first impedances to measure the effect of the biological reagent inducing metabolic deviations to the biological sample in the target chamber and determine whether said biological sample is sensitive to said biological reagent.

57. A method for rapid detection of metabolic deviations comprising:
measuring, at a first time, a first plurality of impedances of a biological sample in a control chamber and a biological reagent and the biological sample in a target chamber;
measuring, at a second time, a second plurality of impedances of the biological sample in the control chamber and the biological reagent and the biological sample in the target chamber; and
comparing the measured second plurality of impedances to the measured first plurality of impedances to measure the effect of the biological reagent inducing metabolic deviations to the biological sample in the target chamber and determine whether said biological sample is sensitive to said biological reagent.
